(19) United States
(12) Patent Application Publication
(10) Pub. No.: US 2019/0233900 A1
(43) Pub. Date: Aug. 1, 2019

(54) BIOMARKERS IN DE NOVO PYRIMIDINE SYNTHESIS PATHWAYS AND CHEMORESISTANCE

(71) Applicants: Nantomics, LLC, Culver City, CA (US); NantBio, Inc., Culver City, CA (US)

(72) Inventors: Kevin B. Givechian, Culver City, CA (US); Chad Garner, Culver City, CA (US); Hermes J. Garban, Culver City, CA (US); Shahrooz Rabizadeh, Culver City, CA (US); Patrick Soon-Shiong, Culver City, CA (US)

(21) Appl. No.: 16/264,379

(22) Filed: Jan. 31, 2019

Related U.S. Application Data
(60) Provisional application No. 62/625,244, filed on Feb. 1, 2018.

(51) Int. Cl. C12Q 1/6886; A61K 33/243

(52) U.S. Cl. CPC C12Q 1/6886; A61K 33/243; C12Q 2600/112; C12Q 2600/106; C12Q 2600/156; C12Q 2600/158

(57) ABSTRACT

Compositions, methods, and uses of de novo pyrimidine synthesis pathway element, CAD, and optionally a second gene in a nucleotide excision repair pathway, POLD2 in determining a predicted survival rate, predicted responsiveness to a cisplatin-based chemotherapy of a patient diagnosed with bladder urothelial carcinoma are provided.

This application claims priority to copending US provisional patent application with the Ser. No. 62/625,244, filed Feb. 1, 2018, which is incorporated by reference in its entirety herein.

### FIELD OF THE INVENTION

The field of the invention is biomarkers in DNA repair mechanisms and their use to predict survival rate of cancer patients.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Certain DNA repair gene alterations have recently emerged as a tool to help better stratify urothelial cancer patients into responders and non-responders to systemic chemotherapy. Most commonly, first-line systemic chemotherapy for urothelial carcinoma, as with other cancers, is used to trigger apoptosis in rapidly proliferating cells by forming DNA adducts that interfere with DNA replication and transcription. Accordingly, somatic gene alterations that render DNA repair enzymes defective are associated with improved response to systemic chemotherapy and survival. While mutations in the genes of these repair pathways have been implicated in patient prognosis and response to platinum-based chemotherapy, the complementary analysis of DNA repair and nucleotide supply remains relatively unexplored in urothelial carcinoma.

Nucleotide production includes many complex biochemical processes that are intertwined with feedback mechanisms to appropriately adapt to the metabolic needs of a cell. In regards to chemotherapy response, more recent work has specifically highlighted the ability of cancer cells to exploit the adaptive nature of the de novo pyrimidine synthesis pathway for their own benefit. This pathway was found to be inducible by chemotherapy in triple-negative breast cancer, where targeting the pathway in a combination therapy rendered cancer cells sensitive to chemotherapy. However, despite the potential implication of carbamoyl-phosphate synthetase 2 (CAD, aspartate transcarbamylase or dihydroorotase) during aspartate diversion, the prevalence of DNA repair alterations during chemotherapy treatment, and the activation of de novo NAD+ synthesis for DNA repair during tumor progression (all of which were observed in bladder cancer), the de novo pyrimidine synthesis pathway has not yet been clinically explored in many cancers, including bladder urothelial carcinoma.

A recent study showed that of 21 TCGA (The Cancer Genome Atlas) cancer cohorts, bladder urothelial carcinoma was the only cancer to be statistically associated with DNA repair alterations. This association was rooted in nucleotide excision repair (NER) gene mutations, which were found in at least some cases to be correlated with favorable survival and response to systemic chemotherapy. At the level of differential gene expression, prognostic studies of the various NER genes in bladder urothelial carcinoma are promising albeit few. To this end, analysis of de novo pyrimidine synthesis gene expression and their prognostic value in bladder urothelial carcinoma has been seemingly overlooked to date.

Therefore, even though some associations of DNA repair mechanism with cancer prognosis are known, de novo pyrimidine synthesis pathway in relation to cancer prognosis remained largely unexplored. Thus, there remains a need for improved compositions, methods for and uses of de novo pyrimidine synthesis pathway element(s) in determining cancer prognosis and providing treatment regimens.

### SUMMARY OF THE INVENTION

The inventive subject matter is directed to various compositions of, methods for, and uses, in which genes in the de novo pyrimidine synthesis pathway, and optionally in the nucleotide excision repair pathway, are analyzed to predict responsiveness to a chemotherapy and/or survival rate of a patient having a cancer. Thus, one aspect of the subject matter includes a method of predicting a survival rate of a patient diagnosed with a cancer. In this method, omics data for a tumor cell from the patient is obtained. From the omics data, expression levels in the tumor cell of a first gene in a de novo pyrimidine synthesis pathway and optionally a second gene in a nucleotide excision repair pathway are determined. Then, the survival rate of the patient can be determined based on the expression level of the first gene and optionally the second gene. Most typically, the first gene in the de novo pyrimidine synthesis pathway is CAD, and the second gene in the nucleotide excision repair pathway is POLD2, and/or the expression levels of the first and second genes are determined by measuring mRNA quantities of the first gene and optionally the second gene. Generally, the survival rate of the patient is determined low when the expression levels of the first and second genes are both high, and the survival rate of the patient is determined high when the expression levels of the first and second genes are both low. In some embodiments, the survival rate of the patient is associated with a resistance to a cisplatin-based chemotherapy.

In some embodiments, the method further comprises a step of determining an alteration of the second gene by identifying a missense mutation or a nonsense mutation in the second gene.

Preferably, the first and second genes are selected by identifying a relationship with an overall survival rate with the first and second genes in a group of patients having the cancer using a Cox Proportional-Hazards model. In such embodiment, it is contemplated that the first and second genes are associated with the overall survival rate at p<0.05, and/or expression levels of the first gene of the group of patients is plotted relative to survival rates of the group of patients in Kaplan-Meier plot.

In another aspect of the inventive subject matter, the inventors contemplate a method of predicting a patient's responsiveness to chemotherapy. In this method, omics data for a tumor cell from the patient is obtained. From the omics data, expression levels in the tumor cell of a first gene in a de novo pyrimidine synthesis pathway and optionally a second gene in a nucleotide excision repair pathway are determined. Then, a predicted patient's responsiveness to chemotherapy can be determined based on the expression level of the first gene and optionally the second gene. Typically, the predicted patient's responsiveness to the chemotherapy is a resistance to a cisplatin-based chemotherapy. It is generally contemplated that the predicted patient's responsiveness to the chemotherapy is low when the expression levels of the first and second genes are both high, and the predicted patient's responsiveness to the chemotherapy is high when the expression levels of the first and second genes are both low. In some embodiments, the cancer is at least one of bladder urothelial carcinoma, a liver cancer, and a renal cancer, and/or the predicted patient's responsiveness to the chemotherapy is a resistance to a cisplatin-based chemotherapy.

Most typically, the first gene in the de novo pyrimidine synthesis pathway is CAD, and the second gene in the nucleotide excision repair pathway is POLD2, and/or the expression levels of the first and second genes are determined by measuring mRNA quantities of the first gene and optionally the second gene.

In some embodiments, the method further comprises a step of determining an alteration of the second gene by identifying a missense mutation or a nonsense mutation in the second gene. Preferably, the first and second genes are selected by identifying a relationship with an overall survival rate with the first and second genes in a group of patients having the cancer using a Cox Proportional-Hazards model. In such embodiment, it is contemplated that the first and second genes are associated with the overall survival rate at p<0.05, and/or expression levels of the first gene of the group of patients is plotted relative to survival rates of the group of patients in Kaplan-Meier plot.

In still another aspect of the inventive subject matter, the inventors contemplate a method of providing a treatment regimen for a patient diagnosed with a cancer. Preferably, the cancer is one of bladder urothelial carcinoma, a liver cancer, and a renal cancer. In this method, omics data for a tumor cell from the patient is obtained. From the omics data, expression levels in the tumor cell of a first gene in a de novo pyrimidine synthesis pathway and optionally a second gene in a nucleotide excision repair pathway are determined. Then a treatment regimen can be provided based on the expression level of the first gene and optionally the second gene. Most typically, the treatment regimen is a cisplatin-based chemotherapy, and based on the expression level of the first gene and optionally the second gene, a predicted resistance to a cisplatin-based chemotherapy can be determined. For example, the predicted resistance to a cisplatin-based chemotherapy is high when the expression levels of the first and second genes are both high and the predicted resistance to a cisplatin-based chemotherapy is low when the expression levels of the first and second genes are both low. Thus, when the predicted resistance to a cisplatin-based chemotherapy is low, cisplatin-based chemotherapy can be provided (recommended) as a treatment regime for the patient.

Most typically, the first gene in the de novo pyrimidine synthesis pathway is CAD, and the second gene in the nucleotide excision repair pathway is POLD2, and/or the expression levels of the first and second genes are determined by measuring mRNA quantities of the first gene and optionally the second gene.

In some embodiments, the method further comprises a step of determining an alteration of the second gene by identifying a missense mutation or a nonsense mutation in the second gene. Preferably, the first and second genes are selected by identifying a relationship with an overall survival rate with the first and second genes in a group of patients having the cancer using a Cox Proportional-Hazards model. In such embodiment, it is contemplated that the first and second genes are associated with the overall survival rate at p<0.05, and/or expression levels of the first gene of the group of patients is plotted relative to survival rates of the group of patients in Kaplan-Meier plot.

Still another aspect of the inventive subject matter includes a method of analyzing gene expression in a patient diagnosed with bladder urothelial carcinoma. In this method, omics data for a tumor cell from the patient is obtained. From the omics data, expression levels in the tumor cell of a first gene in a de novo pyrimidine synthesis pathway and optionally a second gene in a nucleotide excision repair pathway are determined. Most typically, the first gene in the de novo pyrimidine synthesis pathway is CAD, and the second gene in the nucleotide excision repair pathway is POLD2, and the expression levels of the first and second genes are determined by measuring mRNA quantities of the first gene and optionally the second gene. Alternatively, an alteration of the second gene, other than expression level, can be determined by identifying a missense mutation or a nonsense mutation in the second gene.

In some embodiments, the method may further comprise a step of determining a predicted patient's responsiveness to a chemotherapy based on the expression level of the first gene and optionally the second gene. In such embodiment, it is preferred that the treatment regimen is a cisplatin-based chemotherapy, and the predicted resistance to a cisplatin-based chemotherapy is high when the expression levels of the first and second genes are both high, and/or the predicted resistance to a cisplatin-based chemotherapy is low when the expression levels of the first and second genes are both low.

Alternatively and/or additionally, the method may further comprise a step of determining the survival rate of the patient based on the expression level of the first gene and optionally the second gene. In such embodiment, the survival rate is determined low when the expression levels of the first and second genes are both high, and/or the survival rate is determined high when the expression levels of the first and second genes are both low.

In some embodiments, the first and second genes are selected by identifying a relationship with an overall survival rate with the first and second genes in a group of patients having the cancer using a Cox Proportional-Hazards model. In such embodiment, the first and second genes are associated with the overall survival rate at P<0.05, and/or expression levels of the first gene of the group of patients is plotted relative to survival rates of the group of patients in Kaplan-Meier plot.

Still another aspect of the inventive subject matter includes a method of treating patient diagnosed with a cancer. This method comprises steps of obtaining omics data for a tumor cell from the patient, determining expression levels in the tumor cell of a first gene in a de novo pyrimidine synthesis pathway and optionally a second gene in a nucleotide excision repair pathway, and treating the patient with a treatment regimen, wherein the treatment regimen is determined based on the expression level of the first gene and optionally the second gene. Typically, the cancer is at least one of bladder urothelial carcinoma, a liver cancer, and a renal cancer, and/or the first gene is CAD, and/or the second gene is POLD2. Preferably, the expression levels are determined by measuring mRNA quantities of mRNA of the first gene and optionally the second gene.

In some embodiments, the method further comprises a step of determining an alteration of the second gene by identifying a missense mutation or a nonsense mutation in the second gene. Alternatively and/or additionally, the method further comprises a step of determining a predicted patient's resistance to a chemotherapy based on the expression level of the first gene and optionally the second gene. In such embodiments, the predicted resistance to a cisplatin-based chemotherapy is high when the expression levels of the first and second genes are both high, and/or the predicted resistance to a cisplatin-based chemotherapy is low when the expression levels of the first and second genes are both low.

In some embodiments, the method may further comprise a step of determining a survival rate of the patient based on the expression level of the first gene and optionally the second gene. In such embodiments, the survival rate is determined low when the expression levels of the first and second genes are both high and/or the survival rate is determined high when the expression levels of the first and second genes are both low.

Preferably, the first and second genes are selected by identifying a relationship with an overall survival rate with the first and second genes in a group of patients having the cancer using a Cox Proportional-Hazards model. In such embodiment, it is also preferred that the first and second genes are associated with the overall survival rate at P<0.05, and/or expression levels of the first gene of the group of patients is plotted relative to survival rates of the group of patients in Kaplan-Meier plot.

Typically, the treatment regimen is a cisplatin-based chemotherapy. In such embodiment, the patient is treated with the cisplatin-based chemotherapy when the expression level of both of the first gene and the second gene are below first and second predetermined thresholds for the first gene and the second gene, respectively. Alternatively, the patient is treated with the cisplatin-based chemotherapy when the expression level of the first gene is below a predetermined threshold, and/or the patient is treated with the cisplatin-based chemotherapy based on the predicted resistance, wherein the predicted resistance calculated based on the expression level of both of the first gene and the second gene. In some embodiments, the method may further comprise a step of determining effectiveness of the treatment regime based on expression levels of the first gene and optionally the second gene measured during or after treating the patient.

Various objects, features, aspects and advantages of the inventive subject matter will become more apparent from the following detailed description of preferred embodiments and accompanied drawings.

### BRIEF DESCRIPTION OF THE DRAWING

FIG. 1A illustrates a workflow for identifying gene signatures of de novo pyrimidine synthesis pathway and nucleotide excision repair pathway related to overall survival rate of bladder urothelial carcinoma patients.

FIG. 1B illustrates a de novo pyrimidine synthesis pathway.

FIG. 2A-D show Kaplan-Meier curves for individual prognostic effect of CAD gene expression related to overall survival rate in bladder urothelial cancer patients. FIG. 2A shows that high expression of CAD was associated with poor prognosis (P=0.008) in the discovery dataset. FIG. 2B shows that high expression of CAD was associated with poor prognosis in the validation dataset (P=0.017). FIG. 2C shows CAD expression relative to low/high risk group in the discovery set (P<0.001). FIG. 2D shows CAD expression relative to low/high risk group in the validation set (P<0.001).

FIG. 3A-D show Kaplan-Meier curves for individual prognostic effect of POLD2 gene expression related to overall survival rate in bladder urothelial cancer patients. FIG. 3A shows that high expression of POLD2 was associated with poor prognosis (P=0.023) in the discovery dataset. FIG. 3B shows that high expression of POLD2 was associated with poor prognosis in the validation dataset (P=0.019). FIG. 3C shows POLD2 expression relative to low/high risk group in the discovery set (P<0.001). FIG. 3D shows POLD2 expression relative to low/high risk group in the validation set (P<0.001).

FIG. 4A-F show CAD/POLD2 expression analysis and independent association with drug response. CAD/POLD2 expression was associated with poor prognosis in the discovery dataset (P=0.014) (shown in FIG. 4A) and the validation dataset (P=0.043) (shown in FIG. 4B). CAD/POLD2 expressions relative to low/high risk group in the discover set (P<0.001) (shown in FIG. 4C). CAD/POLD2 expressions relative to low/high risk group in the validation set (P 0.001) (shown in FIG. 4d). Multivariate model results of CAD/POLD2 expression cohorts at full duration patient follow-up (Logrank P=0.0019) (shown in FIG. 4E) and at 1,500-day patient follow-up (Logrank P=1.16e-5) (shown in FIG. 4F).

FIG. 5A-F show bar graphs of drug responses in relation to CM) or POLD2 expressions. CAD/POLD2 expression was associated with poor prognosis in the discovery dataset (P=0.014) (shown in FIG. 5A) and the validation dataset (P===0.043) (shown in FIG. 5A). CAD/POLD2 expressions relative to low/high risk group in the discover set (P<0.001) (shown in FIG. 5C). CAD/POLD2 expressions relative to low/high risk group in the validation set (P<0.001) (shown in FIG. 51)). Multivariate model results of CAD/POLD2 expression cohorts at full duration patient follow-up (Logrank P=0.0019) (shown in FIG. 5E) and at 1,500-day patient follow-up (Logrank P=1.16e-5) (shown in FIG. 5F).

FIG. 6 shows a heat map describing hierarchal clustering of the 17 co-alteration/mutual-exclusive NER genes and CAD to visualize clusters by patient expression z-scores. Columns correspond to TCGA patients and rows correspond to genes.

FIG. 7 shows OncoPrint results from cBioPortal for CAD and 17 co-altered/mutually-exclusive NER genes in TCGA samples.

### DETAILED DESCRIPTION

Somatic mutations in DNA repair genes have in certain cases been clinically associated with chemosensitivity, although few studies have interrogated the nucleotide synthesis pathways that supply DNA repair processes. Previous work suggested that bladder urothelial carcinoma is uniquely enriched for mutations in nucleotide excision repair genes, and that these mutations may be associated with response to platinum-based therapy and favorable survival. Conversely, the de novo pyrimidine synthesis pathway has recently emerged as a putative clinical target. This anabolic process is thought to supply DNA repair processes such as nucleotide excision repair; that is, DNA repair enzymes may require a sufficient nucleotide supply available to reverse the intended genotoxic damage of systemic chemotherapy in rapidly proliferating cancer cells.

Owing to the relatively unexplored de novo pyrimidine synthesis pathway in cancer, the inventors explored the prognostic complementarity between de novo pyrimidine synthesis and nucleotide excision repair expression in a total of 570 bladder urothelial carcinoma patients. De novo pyrimidine synthesis gene expression and potentially complementary nucleotide excision repair pathway gene expression were analyzed by multifactorial prognostic analysis. With such analysis, the inventors now discovered that de novo pyrimidine synthesis gene CAD is associated with unfavorable overall survival and is co-altered with seventeen genes involved in the nucleotide excision repair pathway. Among the seventeen genes in the nucleotide excision repair pathway, the inventors further discovered that POLD2 expression is directly associated with worse overall survival rate.

Thus, the inventors discovered that a survival rate of a patient diagnosed with a cancer, more specifically bladder urothelial carcinoma, can be predicted by obtaining omics data for a tumor cell from the patient and determining the expression levels of a gene in the de novo pyrimidine synthesis pathway and/or a gene in the nucleotide excision repair pathway that preferably is co-altered with the gene in the de novo pyrimidine synthesis pathway.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

Obtaining Omics Data

Any suitable methods of obtaining omics data for the tumor cell from the patient (or healthy tissue from a patient or a healthy individual as a comparison) are contemplated. In some embodiments, a step of obtaining omics data includes a step of obtaining a tumor tissue or a tumor cell from the patient (or healthy tissue from a patient or a healthy individual as a comparison), preferably via a biopsy (including liquid biopsy, or obtained via tissue excision during a surgery, etc.). The tumor cells or tumor tissue (or tissues) may include cells and/or tissues from a single or multiple different tissues or anatomical regions. For example, the omics data can be obtained from a pancreatic cancer cell in the patient's pancreas (and/or nearby areas for metastasized cells), and a normal pancreatic cells (non-cancerous cells) of the patient or a normal pancreatic cells from a healthy individual other than the patient. Also, the tumor cells or tumor tissue (or tissues) may include any unprocessed or processed tissues or cells. For example, the tumor cells or tumor tissue may be fresh or frozen. For other example, the tumor cells or tumor may be in a form of cell/tissue extracts. From the obtained tumor cells or tumor tissue, DNA, RNA (e.g., mRNA, miRNA, siRNA, shRNA, etc.), and/or proteins (e.g., membrane protein, cytosolic protein, nucleic protein, etc.) can be isolated and further analyzed to obtain omics data. In other embodiments, a step of obtaining omics data may include receiving omics data from a database that stores omics information of one or more patients and/or healthy individuals.

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Moreover, it should be noted that the data sets are preferably reflective of a tumor and a matched normal sample of the same patient to so obtain patient and tumor specific information. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

With respect to the analysis of tumor and matched normal tissue of a patient, numerous manners are deemed suitable for use herein so long as such methods will be able to generate a differential sequence object or other identification of location-specific difference between tumor and matched normal sequences. However, it is especially preferred that the differential sequence object is generated by incremental synchronous alignment of BAM files representing genomic sequence information of the diseased and the matched normal sample. For example, particularly preferred methods include BAMBAM-based methods as described in US2012/0059670A1 and US20120066001A1.

In addition, omics data of cancer and/or normal cells comprises transcriptome data set that includes sequence information and expression level (including expression profiling or splice variant analysis) of RNA(s) (preferably cellular mRNAs) that is obtained from the patient, most preferably from the cancer tissue (diseased tissue) and matched healthy tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

It should be appreciated that one or more desired nucleic acids may be selected for a particular disease, disease stage, specific mutation, or even on the basis of personal mutational profiles or presence of expressed neoepitopes. Alternatively, where discovery or scanning for new mutations or changes in expression of a particular gene is desired, real time quantitative PCR may be replaced by RNAseq to so cover at least part of a patient transcriptome. Moreover, it should be appreciated that analysis can be performed static or over a time course with repeated sampling to obtain a dynamic picture without the need for biopsy of the tumor or a metastasis.

Further, omics data of cancer and/or normal cells comprises proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)). Consequently, it should be appreciated that the above methods will provide patient and tumor specific neoepitopes, which may be further filtered by sub-cellular location of the protein containing the neoepitope (e.g., membrane location), the expression strength (e.g., overexpressed as compared to matched normal of the same patient), etc.

Gene Expressions and Implications in Survival Rate and Resistance to Chemotherapy

While it is contemplated that any relevant omics data can be used to determine an association between de novo pyrimidine synthesis pathway element (and/or nucleotide excision repair pathway) and cancer prognosis (especially bladder urothelial carcinoma), the inventors found that at least some mRNA expression levels of pyrimidine synthesis pathway element(s) (and/or nucleotide excision repair pathway element(s)) can be strongly associated with overall survival rate of the cancer patients.

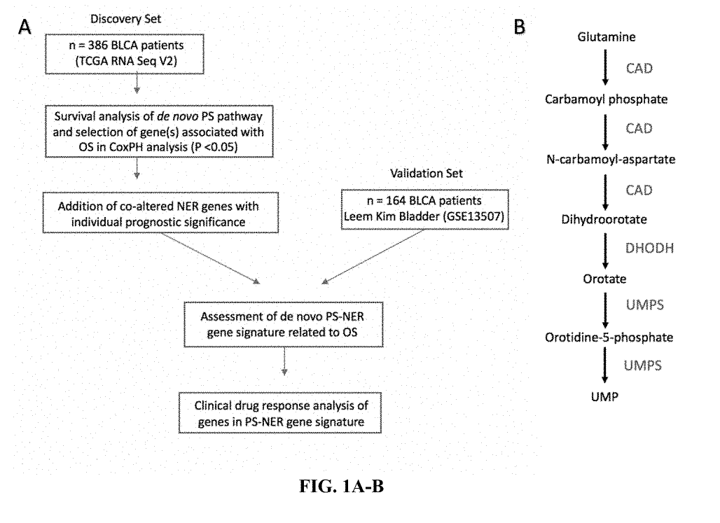

For example, as shown in FIG. 1A-B, the inventors obtained two sets of patient samples or data, 1) discovery set, and 2) validation set. In the discovery set, the inventors examined 386 patient primary tumor samples with available clinical survival data and RNA-seq V2 expression data in the TCGA bladder urothelial carcinoma 2016 dataset via SurvExpress (clinical characteristics available at firebrowse.org). These patients were evaluated for overall survival relative to primary tumor gene expression. In the validation set, the inventors examined 164 primary patient bladder cancer samples that were expression profiled by array (clinical characteristics available via GEO accession GSE13507). The workflow using the discovery set, and validation set is described in FIG. 1A.

De Novo Pyrimidine Synthesis Genes Related to Overall Survival Rate:

Gene expression was evaluated to determine those strongly associated with overall survival rate (P<0.05) using a CoxPH model in R via SurvExpress to determine hazard ratio relative to the risk group. The data of each set was the original (quantile-normalized) data, and for the validation microarray set, all probe sets were averaged per sample (e.g., if multiple probe sets existed for a gene). Samples were ordered according to prognostic index (PI) each patient and separated into risk group cohorts by a median split. The formula used to generate the prognostic index is below:

PI=βx

where β can be interpreted as a risk/linear regression coefficient for x, which is the expression value for a gene of interest in a given tumor sample. β for each gene was obtained from the Cox fitting. OS was shown by Kaplan-Meier (KM) plots. KM Plots were generated with cohorts segregated by risk groups by the PI median relative to high versus low gene expression, and survival curves were generated and compared using the log-rank test.

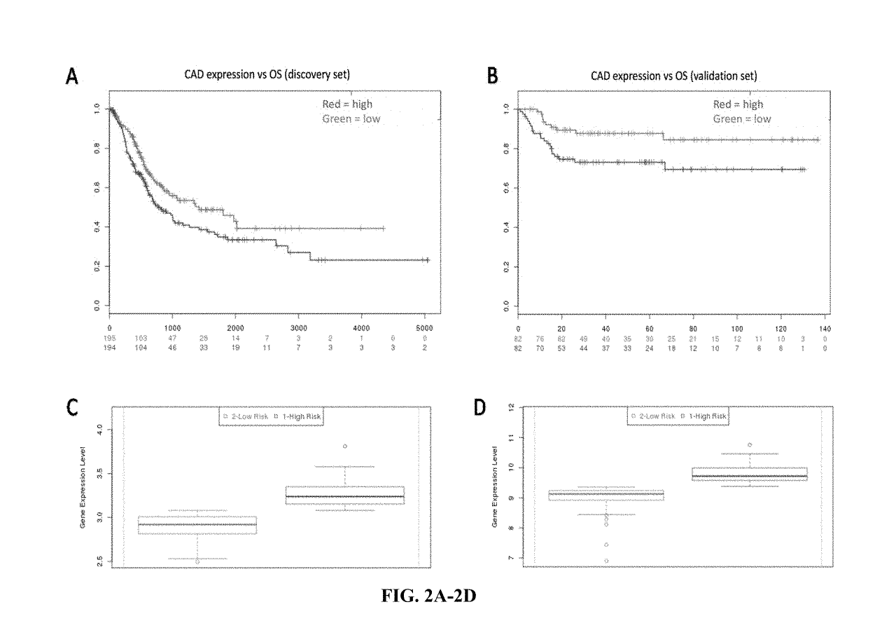

Among the transcriptome data, the inventors selected de novo pyrimidine synthesis pathway genes and nucleotide excision repair pathway genes for initial analysis to associate with overall survival rate of patients. The de novo pyrimidine synthesis pathway is described in FIG. 1B. The nucleotide excision repair pathway gene set used for preliminary analysis was the Kegg Nucleotide Excision Repair pathway (hsa03420), which contained 44 genes. Of the three genes in the de novo pyrimidine synthesis pathway, the inventors discovered that CAD was most strongly associated with poor survival rate in the discovery set (P=0.008; HR=1.44, 95% CI: 1.06-1.95; as shown in Table 1). The prognostic significance of CAD was confirmed in the validation set (P=0.017; HR=2.42, 95% CI: 1.14-5.11; as shown in Table 1). Kaplan-Meier plots show the prognostic effect of CAD expression in the discovery and validation sets (FIG. 2A-B, respectively). Boxplots show differential gene expression by risk group for CAD in the discovery (P<0.001) and validation set (P<0.001; FIG. 2C-D, respectively).

TABLE 1
Cox proportional hazards model results for de novo pyrimidine synthesis gene expression

| | Discovery (n = 386) | | | | Validation (n = 164) | | | |
|---|---|---|---|---|---|---|---|---|
| | | | | Risk | | | | Risk |
| | HR | P- | Regression | group | HR | | Regression | group |
| Dataset | (95% CI) | value | coefficient | expression | (95% CI) | P-value | coefficient | expression |
| CAD* | 1.44 (1.06-1.95) | 0.008* | 0.977 | high | 2.42 (1.14-5.11) | 0.017* | 0.715 | high |
| DHODH | 1.15 (0.85-1.54) | 0.160 | 0.325 | high | 1.17 (0.58-2.34) | 0.6631 | 1.25 | high |
| UMPS | 1.13 (0.84-1.52) | 0.428 | −0.346 | low | 1.58 (0.78-3.21) | 0.1987 | −0.075 | low |

Abbreviations:
CI, confidence interval;
HR, hazard ratio for risk group;
*indicates significance (P < 0.05).

Analysis of NER Genes Co-Altered with CAD:

In addition to the de novo pyrimidine synthesis pathway genes, genes in the Nucleotide Excision Repair pathway were evaluated for their degree of co-alteration with CAD through the cBioPortal mutual exclusivity and co-occurrence module, using the TCGA Provisional dataset (n=408). P-values were derived from Fisher's exact test. The log odds ratio quantifies how strongly the presence or absence of alterations of two genes is associated in the tumor samples (log odds ratio ≥0 association towards co-occurrence; log odds ratio ≤0 association towards mutual exclusivity). Subsequently, Kegg NER genes selected for prognostic analysis in the discovery and validation set were restricted to those significantly co-altered with CAD. The OncoPrint visualization was generated in cBioPortal, and the unsupervised expression heat map and corresponding denogram were generated in R using the ComplexHeatmap library.

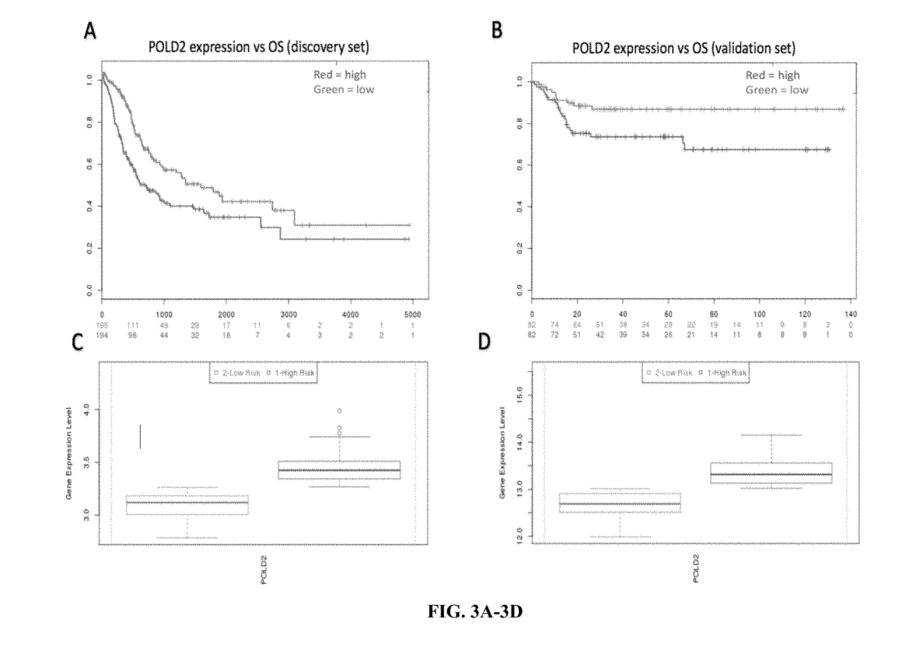

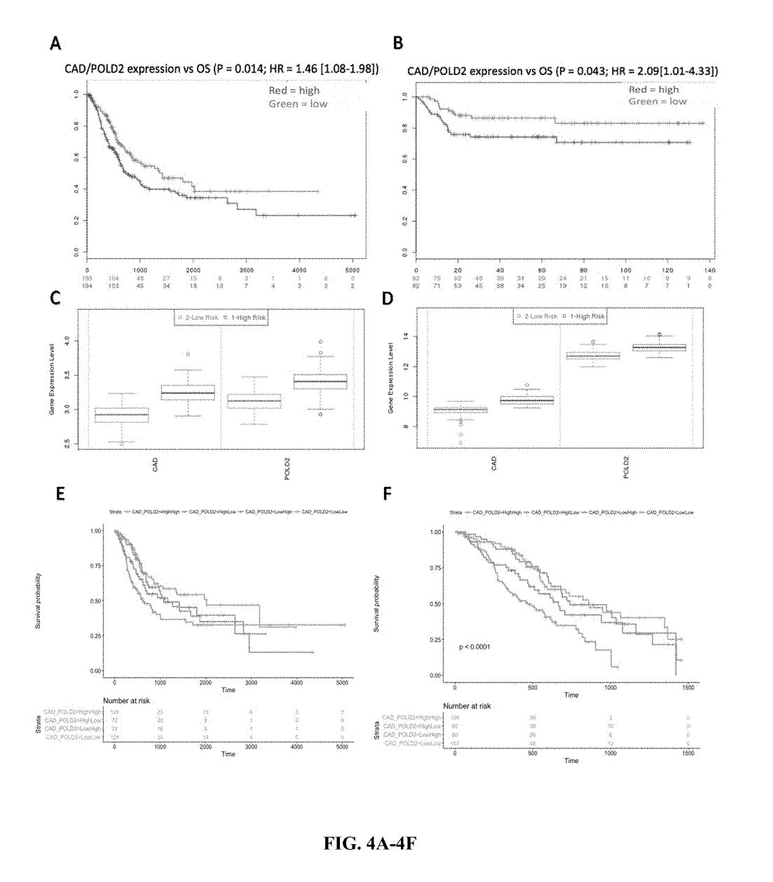

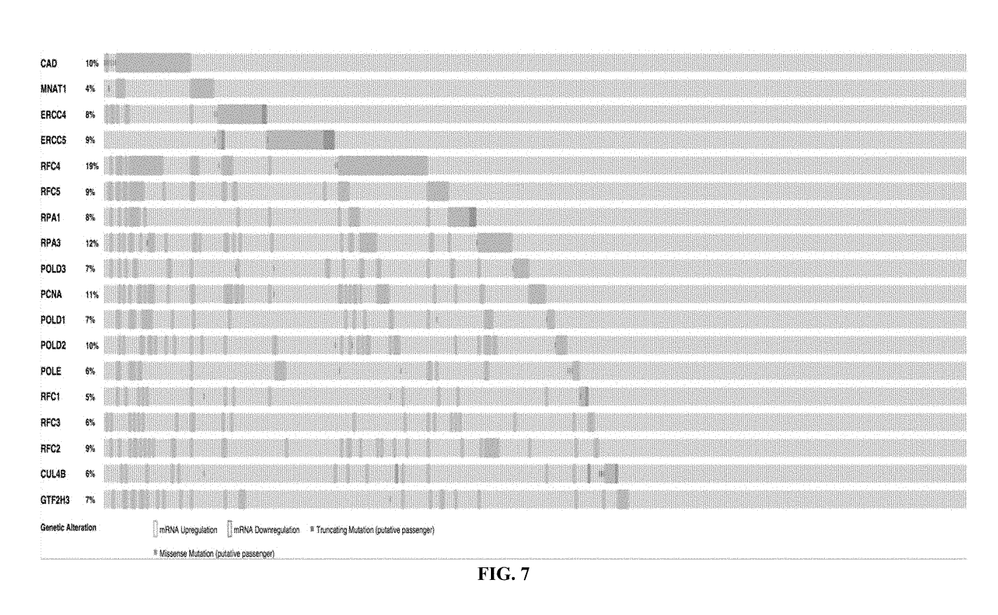

The Kegg Nucleotide Excision Repair gene set was used to analyze which NER genes may be associated with CAD that may also hold prognostic significance. Table 2 shows cBioPortal co-alteration/mutual-exclusivity results for CAD and the genes in the Kegg NER pathway. There were 17 genes involved in NER that were significantly co-altered with CAD. This co-alteration analysis accounted for mRNA upregulation/downregulation, missense mutations, and nonsense mutations. An unsupervised heat map was produced to show expression clusters of CAD and the 17 co-altered Nucleotide Excision Repair genes from cBioPortal (FIG. 7). Each of the 17 CAD-associated Nucleotide Excision Repair genes was analyzed for prognostic significance in the discovery set. Of these 17 Nucleotide Excision Repair genes, ERCC3, ERCC5, and POLD2 each were significantly related to overall survival (OS) (P<0.05; Table 3: Cox model results for each of the CAD-associated Nucleotide Excision Repair genes in the Kegg Nucleotide Excision Repair pathway. ERCC3, ERCC5, and POLD2 each held prognostic significance (P=0.015, P 0.008, P===0.023, respectively)). ERCC3 and ERCC5 had protective effects (risk group expression=low) and were not associated with response to systemic chemotherapy, while only POLD2 expression was associated with unfavorable prognostic effect and drug resistance (risk group expression=high, P=0.023; HR=1.40, 95% CI: 1.04-1.98; FIG. 3A, and FIG. 4D, respectively). ERCC2 and ERCC5 were therefore excluded from further analysis. To validate the prognostic significance of POLD2 expression, overall survival analysis shows POLD2 expression associated with poor survival in the validation dataset (P=0.019; HR=2.38, 95% CI: 1.13-5.03; FIG. 3B). The high-risk group patients in both the discovery set and validation sets possessed higher expression of POLD2 (P<0.001; FIG. 3C-D).

TABLE 2
Nucleotide Excision Repair genes significantly co-altered
with CAD in TCGA bladder urothelial carcinoma dataset

| | | | Log Odds | |
|---|---|---|---|---|
| Gene A | Gene B | p-Value | Ratio | Association |
| CAD | MNAT1 | 0.009172906 | 1.499132138 | Tendency towards co-occurrence |
| CAD | ERCC3 | 0.025363905 | 0.852173175 | Tendency towards co-occurrence |
| CAD | ERCC5 | 0.024046249 | −1.834319084 | Tendency towards mutual exclusivity |
| CAD | RFC4 | 2.22E−06 | 1.542470638 | Tendency towards co-occurrence |
| CAD | RFC5 | 1.05E−05 | 1.746639034 | Tendency towards co-occurrence |
| CAD | RPA1 | 0.033659832 | 0.882500409 | Tendency towards co-occurrence |
| CAD | RPA3 | 0.008758494 | 0.965561781 | Tendency towards co-occurrence |
| CAD | POLD3 | 0.014035124 | 1.073294481 | Tendency towards co-occurrence |
| CAD | PCNA | 0.004450951 | 1.114698426 | Tendency towards co-occurrence |
| CAD | POLD1 | 2.79E−05 | 1.774638428 | Tendency towards co-occurrence |
| CAD | POLD2 | 0.019814681 | 0.944634261 | Tendency towards co-occurrence |
| CAD | POLE | 0.016617056 | 1.109637759 | Tendency towards co-occurrence |
| CAD | RFC1 | 0.009096674 | 1.355262252 | Tendency towards co-occurrence |
| CAD | RFC3 | 0.006386313 | 1.163269511 | Tendency towards co-occurrence |
| CAD | RFC2 | 0.000535725 | 1.438938095 | Tendency towards co-occurrence |
| CAD | CUL4B | 0.023967257 | 1.107345969 | Tendency towards co-occurrence |
| CAD | GTF2H3 | 7.79E−05 | 1.718898121 | Tendency towards co-occurrence |

As indicated by ERCC3 and ERCC 5, not all nucleotide excision repair genes have same types or correlations (e.g., inverse or direct) with respect to overall survival rate of patients. For example, ERCC1 and ERCC2 has opposite effect to the complementation groups (ERCC3 and ERCC5), suggesting a context-dependent clinical effect for varying excision repair complementation groups. Further, not all nucleotide excision repair genes that are co-altered with CAD are not corroborated by drug response analysis, indicating that not all genes in the nucleotide excision repair pathway can be a reliable marker or candidate for a marker to determine or predict survival rate of a patient and/or drug response of the tumor cell.

TABLE 3
Cox proportional hazards model results for co-altered
Nucleotide Excision Repair gene expression

| | Discovery Set | | |
|---|---|---|---|
| Gene | HR (95% CI) | P-value | Regression coefficient |
| MNAT1 | 1.27 (0.94-1.71) | 0.120 | 0.43 |
| ERCC3* | 1.45 (1.07-1.96) | 0.015* | −0.437 |
| ERCC5* | 1.68 (1.24-2.27) | 0.0008* | −1.251 |
| RFC4 | 0.97 (0.72-1.31) | 0.856 | 0.01 |
| RFC5 | 1.15 (0.85-1.55) | 0.356 | −0.217 |
| RPA1 | 1.32 (0.98-1.78) | 0.072 | 0.71 |
| RPA3 | 0.88 (0.65-1.19) | 0.392 | −0.05 |
| POLD3 | 1.03 (0.77-1.39) | 0.828 | 0.025 |
| PCNA | 1.29 (0.96-1.74) | 0.092 | −0.257 |
| POLD1 | 1.12 (0.83-1.52) | 0.440 | −0.306 |
| POLD2* | 1.4 (1.04-1.89) | 0.023* | 0.715 |
| POLE | 1.12 (0.83-1.5) | 0.474 | −0.315 |
| RFC1 | 1.03 (0.76-1.39) | 0.859 | 0.097 |
| RFC3 | 1.05 (0.78-1.42) | 0.732 | 0.122 |
| RFC2 | 0.94 (0.7-1.27) | 0.694 | 0.234 |
| CUL4B | 1.08 (0.8-1.46) | 0.606 | 0.263 |
| GTF2H3 | 1.22 (0.91-1.65) | 0.190 | 0.175 |

Abbreviations:
CI, confidence interval;
HR, hazard ratio for risk group;
*indicates significance (P < 0.05)

Multifactorial Analysis of CAD/POLD2 Expression Related to OS:

For discovery and validation of CAD/POLD2 prognostic significance, regression coefficients were individually obtained from the discovery data set models. These regression coefficients served as the weights in the final CoxPH validation model in which risk group cohorts were separated at the median PI for the cutoff as shown by:

PI_{CAD/POLD2}=β_{CAD}x_{CAD}+β_{POLD2}x_{POLD2}

where β_{CAD}=0.977 and β_{POLD2}=0.715. Patient OS relative to patient expression of CAD/POLD2 was shown by the Kaplan-Meier (KM) method. KM Plots were generated with cohorts segregated by risk groups by the PI median relative to high versus low gene expression of CAD and POLD2 in the final linear model, and survival curves were generated and compared using the KM method and the log-rank test. The survival and survminer packages were used to conduct multivariate Cox regression analysis of the TCGA data set in R with median expression cutoffs.

When combined, CAD and POLD2 gene expression was associated with OS in both the discovery and validation datasets (P=0.014; HR=1.46, 95% CI: 1.08-1.98 and P=0.043; HR=2.09, 95% CI: 1.01-4.43, respectively; FIG. 4A-B). The high-risk group patients (PI>median) in both the discovery set and validation sets possessed higher expression of CAD/POLD2 (P<0.001; FIG. 4C-D). The inventors also fit a multivariate model and showed that patients that possessed both high CAD and high POLD2 expression together exhibited the worst overall survival (Logrank P=0.0019; FIG. 4E, Table 3). It is contemplated that the unfavorable synergism between CAD and POLD2 would be more pronounced early during the course of treatment to ameliorate chemotherapy induced DNA adducts. Thus, the inventors examined patient survival during the first 1,500 days. Indeed, patient overall survival was exacerbated when patient follow-up was restricted to the first 1,500 days (Logrank P=1.16e-5; FIG. 4F, Table 4), suggesting a relatively early unfavorable complementarity between CAD and POLD2 expression.

TABLE 4
Cox proportional hazards multivariate results for CAD and POLD2

| Full patient follow-up | | | | Patient follow-up restricted to 1,500 days | | |
|---|---|---|---|---|---|---|
| | Discovery Set | P- | Regression | Discovery Set | | Regression |
| Gene | HR (95% CI) | value | coefficient | HR (95% CI) | P-value | coefficient |
| POLD2 | 0.66 (0.48-0.89) | 0.007* | −0.42 | 0.52 (0.38-0.73 | 9.81e−5* | −0.64 |
| CAD | 0.78 (0.57-1.05) | 0.106 | −0.25 | 0.72 (0.52-0.98) | 0.040* | −0.33 |

Abbreviations:
CI, confidence interval;
HR, hazard ratio for low-risk/expression group;
* indicates significance (P < 0.05)

CAD and POLD2 Association with Patient Drug Response:

In addition, the inventors further determined associations between CAD and/or POLD2 expression and resistance to systemic chemotherapy. Curated records of drug treatments and outcomes generated from TCGA clinical data were used to analyze the differential gene expressions of bladder urothelial carcinoma patients who were sensitive or resistant to systemic chemotherapy. There was a total of 65 bladder urothelial carcinoma patients with clinical drug-response annotation to systemic chemotherapy and corresponding pre-treatment log 2-normalized RNA-seq V2 expression data (responders: n=37, non-responders: n=28). There was a total of 31 bladder urothelial carcinoma patients with clinical response labels to cisplatin-based therapy and corresponding pre-treatment log 2-normalized mRNA-seq expression data (responders: n=22, non-responders: n=9). Two-tailed t-tests were used to determine differential expression significance, and Pearson r values were calculated for log 2-normalized patient gene expression correlation analysis.

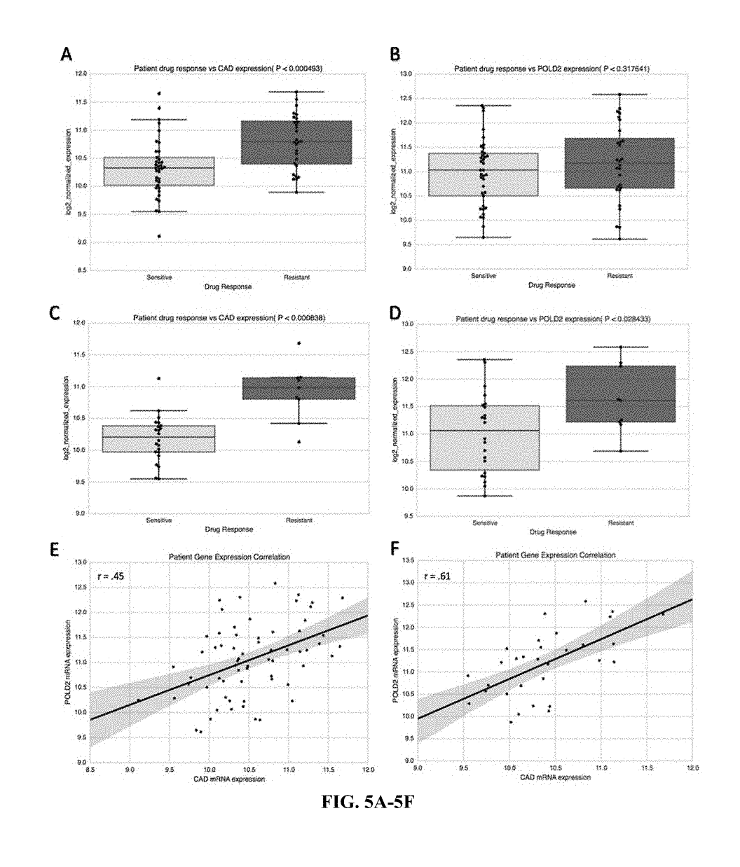

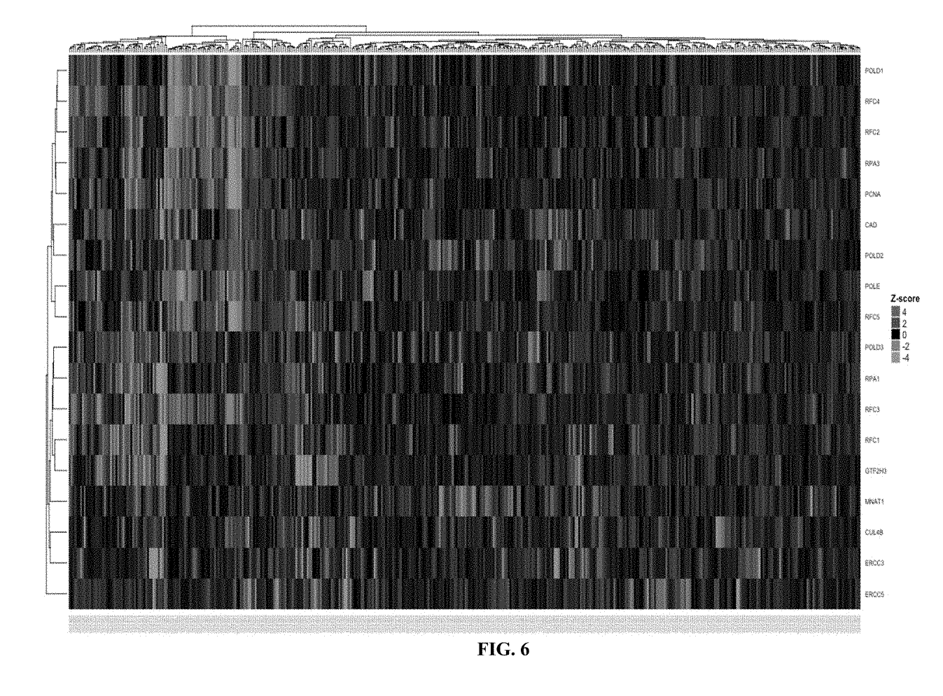

When CAD and POLD2 were examined for their association with drug response data in bladder urothelial carcinoma, CAD expression associated with resistance to systemic chemotherapy (P=4.93e-4; FIG. 5A), but this did not hold true for POLD2 (P=0.318; FIG. 5B). Interestingly, however, POLD2 has been implicated in cellular resistance specifically to cisplatin, due to its ability to dramatically increase the efficiency and processivity of DNA synthesis via interaction with Pol ζ4 in order to bypass 1,2-intrastrand d(GpG)-cisplatin cross-links. In light of this, the inventors examined whether the unfavorable prognostic effects of POLD2 may instead be specifically through resistance to cisplatin-based therapy, which is a standard first-line therapy in bladder urothelial carcinoma. In patients treated with cisplatin-based therapy, CAD and POLD2 were both significantly associated with cisplatin-based therapy resistance (P=8.38e-4 and P=0.028, respectively; FIG. 5C-D), suggesting that, unlike for CAD, the chemoresistant effects of POLD2 may be specific to cisplatin-based therapy. To determine the extent to which CAD and POLD2 patient expressions were correlated in samples of the drug response analysis, the inventors examined Pearson correlation coefficients. When restricted to patients administered cisplatin-based therapy, patient expressions became more tightly correlated (r=0.40, P<0.001 vs r=0.60, P<0.001, respectively; FIG. 5E-F).

Without wishing to be bound to any specific theory, the inventors contemplate that CAD is strongly associated with survival rate of bladder urothelial carcinoma patients as CAD catalyzes the first three steps of de novo pyrimidine synthesis pathway, in contrast to proceeding two genes of the de novo PS pathway. DHODH and UMPS. DHODH and UMPS, are not significantly associated with overall survival rate, and they independently catalyze fewer steps of the pathway. Intriguingly, CAD is also associated with unfavorable survival in liver cancer and renal cancer, and it catalyzes the rate-limiting step of the de novo pyrimidine synthesis pathway, suggesting it may be expressed at higher levels than DHODH and UMPS in de novo pyrimidine synthesis to ameliorate chemotherapy induced genotoxic damage. The inventors' prognostic observations of CAD are also in line with its amplification as a marker of genomic instability in tumorigenic liver cells, its association with mutant TP53 status, and its implication in cancer cell viability in bladder urothelial carcinoma and triple negative breast cancer. Thus, it is further contemplated that objective catalytic involvement of CAD in pyrimidine production may in part be to supply nucleotide excision repair enzymes, the re-building blocks necessary to repair genotoxic damage from systemic chemotherapy, as has been demonstrated in the context of DNA replication. Providing sufficient nucleotides for nucleotide excision repair may in turn mitigate the intended pro-apoptotic effects of chemotherapeutic compounds, offering a biological explanation for the inventors' prognostic observations.

With respect to POLD2, POLD2 is a subunit of the DNA polymerase delta exonuclease complex and is known to play a crucial role in nucleotide excision repair. Additionally, POLD2 has been implicated in ovarian carcinogenesis as well as poor glioma patient prognosis. This catalytic subunit has also been associated with poor survival in serous carcinoma, as well as 1,2-intrastrand d(GpG)-cisplatin cross-link bypass via improved Pol ζ efficiency and cooperativity. Thus, it is contemplated that higher expressions of POLD2 and CAD ameliorate pro-apoptotic cisplatin-based therapy DNA damage by bypassing cisplatin-induced DNA adducts and maintain a sufficient pyrimidine pool for repair. Of note, multivariate analysis revealed that both CAD and POLD2 expression (which are moderately correlated in the TOGA. BLCA Provisional dataset; r 0.37), were synergistically associated with poor survival during the first 1,500 days of patient follow-up. This may therefore suggest that the detrimental effect of high CAD/POLD2 co-expression is pronounced early in the course of the disease when patients are generally more aggressively treated with systemic chemotherapy regimens such as cisplatin-based therapy. Therefore, it is possible that the unfavorable prognostic effect of CAD/POLD2 co-expression is driven by the ability to suppress the pro-apoptotic effects of chemotherapy.

Thus, the inventors contemplate that the omics data of the tumor cell, especially those related to de novo pyrimidine synthesis pathway element, and/or nucleotide excision repair pathway, preferably POLD2 (when combined with CAD), can be used to determine a predicted survival rate of a patient diagnosed with cancer, preferably bladder urothelial carcinoma, or possibly some types of liver cancer or renal cancer where CAD is associated with survival rates, or some other types of cancer that shares similar molecular characteristics with the bladder urothelial carcinoma (e.g., sharing pathway characteristics, similar mutations, etc.), and/or to determine a predicted response (of a patient with a cancer) to some types of chemotherapy, especially resistance to cisplatin-based chemotherapy, and/or to provide a treatment regimen, especially a recommendation whether a cisplatin-based chemotherapy can be included in the treatment regime to a patient having bladder urothelial carcinoma.

Any omics data of genes or proteins that are significantly associated (e.g., p<0.1, p<0.05, p<0.01, p<0.005, etc.) with overall survival rate of the patients having a cancer are contemplated, and preferred genes or proteins can be selected by identifying a relationship with an overall survival rate with the genes in a group of patients having the cancer using a Cox Proportional-Hazards model. Thus, exemplary and/or preferred genes/proteins include preferably CAD among de novo pyrimidine synthesis pathway element, and/or POLD2 (when combined with CAD) among nucleotide excision repair pathway elements. Most typically, expression levels of CAD and optionally POLD2 in the tumor cell can be determined by measuring mRNA quantities using any suitable techniques including real-time RT-PCR. Preferably, measured mRNA quantities of CAD and/or POLD2 are normalized against one or more reference genes (a housekeeper gene, e.g., GAPDH, Actin, etc.) such that accurate determination of the absolute level of each mRNA species per cell in any sample may not be substantially compromised by variations in tissue cellularity and RNA yield across samples.

It is contemplated that the expression levels of CAD and/or POLD2 is inversely related to an expected or predicted survival rates of patients with cancer, especially with bladder urothelial carcinoma. Thus, generally, the survival rate can be determined low when the expression levels of CAD is high (e.g., 5% lower expected survival rate per 10% increase of CAD expression level, 5% lower expected survival rate per 20% increase of CAD expression level, 5% lower expected survival rate per 30% increase of CAD expression level, etc.) and the survival rate would be determined high when the expression levels of CAD is low (e.g., 5% higher expected survival rate per 10% decrease of CAD expression level, 5% higher expected survival rate per 20% decrease of CAD expression level, 5% higher expected survival rate per 30% decrease of CAD expression level, etc.). Also, the survival rate can be determined low when the expression levels of POLD2 is high (e.g., 5% lower expected survival rate per 10% increase of POLD2 expression level, 5% lower expected survival rate per 20% increase of POLD2 expression level, 5% lower expected survival rate per 30% increase of POLD2 expression level, etc.), and the survival rate would be determined high when the expression levels of POLD2 is low (e.g., 5% higher expected survival rate per 10% decrease of POLD2 expression level, 5% higher expected survival rate per 20% decrease of POLD2 expression level, 5% higher expected survival rate per 30% decrease of POLD2 expression level, etc.). Further, it is also contemplated that where both CAD and POLD2 expression levels are high, the survival rate can be lower than when only one of CAD and POLD2 is high. Thus, CAD^{high}POLD2^{high} patients can be associated with predicted worse survival than CAD^{high}POLD2^{low} and CAD^{low}POLD2^{high} patients.

Generally, and similar to predicted or expected survival rate, the predicted responsiveness (resistance) to cisplatin-based chemotherapy can be determined high when the expression levels of CAD is high (e.g., 5% higher predicted resistance per 10% increase of CAD expression level, 5% higher predicted resistance per 20% increase of CAD expression level, 5% higher predicted resistance per 30% increase of CAD expression level, etc.), and the predicted resistance to cisplatin-based chemotherapy would be determined low when the expression levels of CAD is low. Alternatively, the predicted sensitivity to cisplatin-based chemotherapy can be determined high when the expression levels of CAD is low (e.g., 5% higher predicted sensitivity per 10% decrease of CAD expression level, 5% higher predicted sensitivity per 20% decrease of CAD expression level, 5% higher predicted sensitivity per 30% decrease of CAD expression level etc.), and the predicted resistance to cisplatin-based chemotherapy would be determined low when the expression levels of CAD is low. Also, the predicted resistance to cisplatin-based chemotherapy can be determined high when the expression levels of POLD2 is high, and the predicted resistance to cisplatin-based chemotherapy would be determined low when the expression levels of POLD2 is low (e.g., 5% lower predicted resistance per 10% decrease of POLD2 expression level, 5% lower predicted resistance per 20% decrease of POLD2 expression level, 5% lower predicted resistance per 30% decrease of POLD2 expression level, etc.). Further, it is also contemplated that where both CAD and POLD2 expression levels are high, the predicted resistance to cisplatin-based chemotherapy can be higher than when only one of CAD and POLD2 is low. Thus, CAD^{high}POLD2^{high} patients can be associated with predicted higher resistance to cisplatin-based chemotherapy than CAD^{high}POLD2^{low} and CAD^{low} POLD2^{high} patients. Thus, together, these biomarkers (CAD/POLD2) could help elucidate mechanisms of chemoresistance to further personalize therapeutic strategies in bladder urothelial carcinoma.

In addition, treatment regimen, especially a recommendation whether a cisplatin-based chemotherapy can be included in the treatment regime to a patient having bladder urothelial carcinoma can be determined and/or provided based on the expression levels of CAD and/or POLD2. Generally, cisplatin-based chemotherapy may not be recommended to be included in the treatment regime if the patient is predicted to have high resistance to cisplatin-based chemotherapy. Conversely, cisplatin-based chemotherapy may be recommended to be included in the treatment regime if the patient is predicted to have low resistance to cisplatin-based chemotherapy. Thus, the treatment regimen may not include a cisplatin-based chemotherapy when the expression level of CAD is high, and the treatment regimen may include a cisplatin-based chemotherapy when the expression level of CAD is low. Also, the treatment regimen may not include a cisplatin-based chemotherapy when the expression level of POLD2 is high, and the treatment regimen may include a cisplatin-based chemotherapy when the expression levels of POLD2 are low. Further, it is also contemplated that where both CAD and POLD2 expression levels are high, it is more likely that the treatment regimen may not include cisplatin-based chemotherapy than when only one of CAD and POLD2 is high.

For example, the survival rate of Patient A can be determined by measuring CAD expression level “X” and/or POLD2 expression level “Y”. CAD expression level “X” can be compared with a Kaplan-Meier plot that plots a plurality of patients' data (survival rate and CAD expression level) and POLD2 expression level “Y” can be compared with another Kaplan-Meier plot that plots a plurality of patients' data (survival rate and PODL2 expression level). Then the survival rate of the patient can be determined based on the fit of “X” (or “Y”) in the Kaplan-Meier plot. In some embodiments, CAD expression level “X” and POLD2 expression level “Y” can be compared with Kaplan-Meier plot that plots a plurality of patients' data (survival rate and CAD/POLD2 expression levels (e.g., as shown in FIG. 4E-F). Thus, in such examples, the survival rate of the patient can be determined as an absolute value (either an approximate or most close value, e.g., 6 months, 12 months, etc.). Alternatively, the survival rate of the patient can be determined “high”, “moderate”, or “low” where the expected or predicted survival of the patient belong to top ⅓, middle ⅓, or bottom ⅓ survival by length among all patients having the same type of cancer, respectively.

The inventors also contemplate that omics information other than expression level of de novo pyrimidine synthesis pathway element(s) and/or nucleotide excision repair pathway element(s) can be obtained to corroborate the prediction of the survival rate. For example, mutation information of CAD and/or POLD2, preferably presence of missense or nonsense mutation in POLD2 can be identified from the omics information, and the presence of missense or nonsense mutation in POLD2 along with the increased expression of POLD2 can further confirm the decreased predicted survival rate of the patient with the cancer. Alternatively, the presence of missense or nonsense mutation in POLD2 can be associated with further decreased predicted survival rate of the patient with the cancer (e.g., 10% decreased predicted survival rate with 30% increased POLD2 expression only and 25% decreased predicted survival rate with 30% increased POLD2 expression with presence of a missense mutation of POLD2, etc.).

Optionally, the inventors further contemplate that where the treatment regimen provided or recommended based on CAD and/or POLD2 expression levels include cisplatin-based chemotherapy, the patient can be further treated and/or administered with cisplatin-based chemotherapy within a day, within a week, within a month after the omics data is obtained from the tumor tissue of the patient. Thus, in another aspect of the inventive subject matter, the inventors contemplate a method of treating a mammal or a patient having a tumor. In such method, omics data for a tumor cell from the patient can be obtained, and the expression levels in the tumor cell of a gene in a de novo pyrimidine synthesis pathway and optionally another gene in a nucleotide excision repair pathway can be determined. Based on the expression levels of the de novo pyrimidine synthesis pathway gene and/or the nucleotide excision repair pathway gene (e.g., the expression levels of the de novo pyrimidine synthesis pathway gene and/or the nucleotide excision repair pathway gene are both below the predetermined threshold, at least one of the expression levels of the de novo pyrimidine synthesis pathway gene and/or the nucleotide excision repair pathway gene is below the predetermined threshold, a predicted patient's responsiveness (sensitivity) to a chemotherapy based on the expression levels of the de novo pyrimidine synthesis pathway gene and/or the nucleotide excision repair pathway gene is high or above a predetermined threshold, or a predicted patient's resistance to a chemotherapy based on the expression levels of the de novo pyrimidine synthesis pathway gene and/or the nucleotide excision repair pathway gene is low or below a predetermined threshold, etc.), the patient can be treated with the chemotherapy (e.g., cisplatin-based chemotherapy) in a dose and schedule effective to treat the tumor.

The predetermined threshold for de novo pyrimidine synthesis pathway gene and/or the nucleotide excision repair pathway gene may vary depending on the type of genes in the de novo pyrimidine synthesis pathway and/or the nucleotide excision repair pathway, and also may vary depending on the type and prognosis of disease (e.g., tumor type, size, location), health status of the patient (e.g., including age, gender, etc.). For example, where the de novo pyrimidine synthesis pathway gene is CAD, the predetermined threshold can be between 9-12 (in log 2 normalized value), preferably between 10-11, preferably between 10.5-11, at least 10.3, at least 10.4, at least 10.5, at least 10.6, at least 10.7, at least 10.8, or at least 10.9 (all in log 2 normalized value). In another example, where the nucleotide excision repair pathway gene is POLD2, the predetermined threshold can be between 9-13 (in log 2 normalized value), preferably between 10-12, preferably between 11-12, more preferably between 11.5-12, at least 11, at least 11.1, at least 11.2, at least 11.3, at least 11.4, at least 11.5 or at least 11.6 (all in log 2 normalized value). Alternatively and/or additionally, the predetermined threshold can be any value of CAD and/or POLD2 (or any other de novo pyrimidine synthesis pathway and/or the nucleotide excision repair pathway genes) that separates the sensitive group of patients from resistant group of patients to the cisplatin-based chemotherapy by p<0.2, preferably p<0.1, more preferably p<0.05, most preferably p<0.01.

As used herein, the term “administering” cisplatin-based chemotherapy refers to both direct and indirect administration of the cisplatin-based chemotherapy, wherein direct administration of the cisplatin-based chemotherapy is typically performed by a health care professional (e.g., physician, nurse, etc.), and wherein indirect administration includes a step of providing or making available the formulation to the health care professional for direct administration (e.g., via injection, infusion, oral delivery, topical delivery, etc.).

With respect to dose and schedule of the cisplatin-based chemotherapy, it is contemplated that the dose and/or schedule may vary depending on the type of agent in combination with the cisplatin-based chemotherapy (e.g., other types of chemotherapy, amifostine to decrease nephrotoxicity, etc.), type and prognosis of disease (e.g., tumor type, size, location), health status of the patient (e.g., including age, gender, etc.). In certain embodiments, the dose can be range from 5-50 mg/m²/day IV, 10-40 mg/m²/day IV, 15-30 mg/m²/day IV, preferably 20 mg/m²/day IV for 7 days/cycle, or preferably 5 days/cycle. In some embodiments, the additional cycle of administration of cisplatin-based chemotherapy can be determined based on the patient's serum creatinine level (SCr, e.g., SCr<1.5 mg/dL [<133 micromoles/L]), blood urea nitrogen level (BUN, e.g., BUN<25 mg/dL [<8.93 mmol/L]), or blood cell counts (e.g., WBC>4000/mm³ and/or platelets>100 k/mm³).

In addition, CAD and/or POLD2 expression levels can be further used to determine the effectiveness of, and/or the response by the patient to the cisplatin-based chemotherapy to guide the future treatment regimen for the patient. For example, CAD and/or POLD2 expression levels can be measured and/or determined prior to, during, and after the cisplatin-based chemotherapy. If the CAD and/or POLD2 expression levels changes during and/or after the cisplatin-based chemotherapy in a direction of lower predicted survival rate or increased resistance to the cisplatin-based chemotherapy, such results may lead to a recommendation to stop or not to recommend further cisplatin-based chemotherapy.

The inventors further contemplate that many more aspects of cancer and cancer prognosis can be associated and/or predicted with CAD and/or POLD2 expression levels or even other genes in de novo pyrimidine synthesis pathway and/or a nucleotide excision repair pathway. For example, the aspects of cancer and cancer prognosis may include tumor stage features, lymph node status, as well as progression- and relapse-free survival. In addition, while a large validation dataset with gene expression profiled by a different platform (e.g., RNA-seq V2 vs affymetrix microarrays) to mitigate potential false positives by obtaining the results across different platforms were used, the inventors also contemplate that a large size of clinical data can be also used to conduct simultaneous multivariate analysis for CAD and POLD2. Further, it is also contemplated that a prospective randomized trial, with a cisplatin-free arm and appropriate gene panel for differential expression analysis can be performed to further distinguish the prognostic value versus predictive power of CAD/POLD2 in cisplatin-based therapy resistance.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A method of treating a patient diagnosed with a cancer, comprising:
obtaining omics data for a tumor cell from the patient;
determining expression levels in the tumor cell of a first gene in a de novo pyrimidine synthesis pathway and optionally a second gene in a nucleotide excision repair pathway; and
treating the patient with a treatment regimen, wherein the treatment regimen is determined based on the expression level of the first gene and optionally the second gene.

2. The method of claim 1, wherein the cancer is at least one of bladder urothelial carcinoma, a liver cancer, and a renal cancer.

3. The method of claim 1, wherein the first gene is CAD.

4. The method of claim 1, wherein the second gene is POLD2.

5. The method of claim 1, wherein the expression levels are determined by measuring mRNA quantities of mRNA of the first gene and optionally the second gene.

6. The method of claim 1, further comprising determining an alteration of the second gene by identifying a missense mutation or a nonsense mutation in the second gene.

7. The method of claim 1, further comprising determining a predicted patient's resistance to a chemotherapy based on the expression level of the first gene and optionally the second gene.

8. The method of claim 7, wherein the predicted resistance to a cisplatin-based chemotherapy is high when the expression levels of the first and second genes are both high.

9. The method of claim 7, wherein the predicted resistance to a cisplatin-based chemotherapy is low when the expression levels of the first and second genes are both low.

10. The method of claim 1, further comprising determining a survival rate of the patient based on the expression level of the first gene and optionally the second gene.

11. The method of claim 10, wherein the survival rate is determined low when the expression levels of the first and second genes are both high.

12. The method of claim 10, wherein the survival rate is determined high when the expression levels of the first and second genes are both low.

13. The method of claim 1, wherein the first and second genes are selected by identifying a relationship with an overall survival rate with the first and second genes in a group of patients having the cancer using a Cox Proportional-Hazards model.

14. The method of claim 13, wherein the first and second genes are associated with the overall survival rate at P<0.05.

15. The method of claim 13, wherein expression levels of the first gene of the group of patients is plotted relative to survival rates of the group of patients in Kaplan-Meier plot.

16. The method of claim 1, wherein the treatment regimen is a cisplatin-based chemotherapy.

17. The method of claim 16, wherein the patient is treated with the cisplatin-based chemotherapy when the expression level of both of the first gene and the second gene are below first and second predetermined thresholds for the first gene and the second gene, respectively.

18. The method of claim 16, wherein the patient is treated with the cisplatin-based chemotherapy when the expression level of the first gene is below a predetermined threshold.

19. The method of claim 16, wherein the patient is treated with the cisplatin-based chemotherapy based on the predicted resistance, wherein the predicted resistance calculated based on the expression level of both of the first gene and the second gene.

20. The method of claim 1, further comprising determining effectiveness of the treatment regime based on expression levels of the first gene and optionally the second gene measured during or after treating the patient.
